(19) United States
(12) Patent Application Publication
(10) Pub. No.: US 2006/0052766 A1
(43) Pub. Date: Mar. 9, 2006

(54) Color-coded medical tubes and post-insertion monitoring thereof

(75) Inventor: Ashish R. Patel, Jamaica Plain, MA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Beth Israel Deaconess Medical Center, Inc., Boston, MA (US)

(21) Appl. No.: 10/937,110

(22) Filed: Sep. 8, 2004

(51) Int. Cl. A61M 25/00

(52) U.S. Cl. 604/523

(57) ABSTRACT

The present invention describes color-coded medical tubes and methods of monitoring the insertion and post-insertion of the same.

### BACKGROUND OF THE INVENTION

During the course of an illness patients often require the insertion of medical tubes for a variety of purposes. Such medical tubes include, nasogastric tubes, chest tubes, feeding tubes, drains, foley catheters and central lines. For example, a nasogastric (NG) tube inserted through the nose, down the esophagus and into the stomach can be used for removing the stomach fluids. Clearly proper insertion and monitoring the placement/location of these tubes in the patient is critical. Most medical professionals who insert such tubes are intensely trained and highly skilled in these techniques, but nonetheless, there are problems. Inappropriate insertion can lead to serious medical complications, even death.

Presently, medical tubes that are inserted into patients are made of either clear/colorless polymers, or are of a single color. Some medical tubes have intervals marked with a thin black line, but these lines are not clearly noticeable and, once inserted into the patient, are not visible at all. Often proper insertion of medical tubes must be confirmed or monitored by x-ray causing additional discomfort to the patient.

Even if a medical tube is initially inserted properly, one of the biggest challenges for a medical professional is post-insertion monitoring. Often, medical tubes that are inserted properly are deliberately pulled at by an uncomfortable patient, or dislocated by accident. Some medical tubes, such as chest tubes, are sutured into place to eliminate the possibility of inadvertent dislocation. The problem of post-insertion monitoring is even more apparent with the increase in home care procedures for patients. Many patients are discharged from the hospital with medical tubes still inserted because of the necessity to continue with treatments at home. In such situations a medical professional is not at home with the patient and the burden of monitoring proper tube insertion falls on the patient or an untrained caregiver. Currently there is no way to easily recognize when a medical tube becomes misplaced after the initial insertion.

### SUMMARY OF THE INVENTION

The present invention relates to a medical tube having at least one distinctive color-coded section. The tubes of the present invention, having one, or more than one, colored sections are collectively referred to herein as multi-colored medical tubes notwithstanding the number of color-coded sections the tube may have.

In a particular embodiment of the present invention, the medical tube comprises a tube body (made of the typical polymer/plastic as conventional medical tubes) with at least one color-coded warning zone section and at least one color-coded danger zone section. The warning zone section and the danger zone section are typically different, distinctive, easily identified colors. The tubes of the present invention can have multiple warning zone sections, and even danger zone sections, but typically have one warning zone and one danger zone. Insertion of the medical tube of the present invention is facilitated by visual identification of the color-coded sections as a guide to the insertion location within the patient. For example, with a nasogastric (NG) tube, about first 40 cm of the distal end of the tube is the danger zone section and is color-coded with a color such as red. The medical professional inserting the NG tube knows that the tube must be inserted into the patient at least as far as the color-coded danger zone section.

Color-coded sections such as a warning zone section and a danger zone section are also greatly advantageous for monitoring post-insertion problems with medical tubes. For example, if the warning zone section becomes visible post-insertion, even an untrained caregiver is alerted that the medical tube may need to be adjusted or reinserted. If the danger zone section becomes visible post-insertion, the caregiver or patient is alerted that the medical tube is in immediate need of adjustment or reinsertion and can contact their physician before serious medical damage can occur.

In one embodiment of the present invention, the medical tube comprises a tube body that is made of a polymer composition that is suitable for (e.g., physiologically compatible) insertion into a patient. This medical tube comprises at least one visually distinguishable section that is color-coded as the danger zone. If the danger zone section becomes visible post-insertion, the medical tube is misplaced and needs to be readjusted or reinserted.

In another embodiment of the present invention, the medical tube comprises at least one visually distinguishable danger zone section and at least one visually distinguishable warning zone section. If the warning zone section becomes visible, the medical tube is in danger of serious dislocation and may need to be readjusted or reinserted. For example, the visibility of the warning zone alerts the caregiver of possible danger and the need for medical attention in the near future. If the danger zone section becomes visible, the medical tube is considered to be misplaced and needs immediate readjustment or reinsertion.

In still another embodiment, the medical tube is a nasogastric tube comprising a proximal and distal end/tip with at least one visually distinguishable danger zone section. This danger zone section represents the length of the average human esophagus and is of a visually distinguishable color from the distal insertion tip of the nasogastric tube to about 40 cm along the tube body from the distal insertion tip toward the proximal tip of the tube.

In yet another embodiment, the medical tube is a nasogastric tube comprising at least one visually distinguishable danger zone section wherein the danger zone section extends from the distal insertion tip to about 40 cm from the distal insertion tip along the tube body. The medical tube also has at least one visually distinguishable warning zone section wherein the warning zone section extends from the end of the danger zone section (at about 40 cm) to about 55 cm from the distal insertion tip along the tube body. It is appreciated by those of skill in the art that the lengths of such color-coded sections along a medical tube of the present invention will vary according to the specific use of the medical tube and that the lengths of color-coded divisions for different specific purposes are easily determined. It is also appreciated by those of skill in the art that the color-coded zones of the medical tubes for the pediatric population will be proportionally smaller.

In one embodiment, the present invention relates to a method of inserting a medical tube comprising inserting a medical tube into a patient and monitoring the movement or location of the color-coded sections of the tube to determine proper placement. The method comprises providing a color-coded medical tube and halting insertion of the medical tube into the patient once a color-coded section is no longer visible, thus indicating that the tube has been properly inserted into the patient. For example, a medical professional will insert the tube until the color-coded danger zone and/or the color-coded warning zone is no longer visible outside the patient, indicating proper insertion of the tube within the patient. Once the color-coded section of the medical tube has been inserted, and is no longer visible, insertion of the tube is halted.

In another embodiment, the present invention pertains to a method for post-insertion monitoring of the insertion of a medical tube. The method comprises monitoring a patient to detect a color-coded section of a medical tube and recognizing that the color code indicates that either the warning zone or the danger zone section is visible and that the medical tube is in need of readjustment or reinsertion.

There are several advantages to the medical tubes of the present invention. First, the tube is not functionally altered in any way. The color-coded medical tube does not require external devices such as magnets or radio transmitters in order to monitor insertion or placement. Second, professionals inserting the tube are aided by the color-coding to determine the position of the tube in the patient's body. This will reduce problems of misplacement by even highly trained medical professionals. Third, once the tube is functioning, any change in the tube's position/location in the patient will be clearly visible thus indicating that correction may be, or is, required.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a depiction of the tube body of a medical tube.

FIG. 2 is a color photograph of a nasogastric tube wherein the danger zone section is color-coded red and the warning zone section is color-coded yellow.

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

### DETAILED DESCRIPTION OF THE INVENTION

A description of preferred embodiments of the invention follows.

The present invention pertains to a medical tube, a method of inserting the medical tube and a method for post-insertion monitoring of the medical tube. As used herein the term “medical tube” means any type of tube which may be inserted into a patient's body and is expected to stay undisturbed within the patient, including, but not limited to, nasogastric tubes, chest tubes, feeding tubes, drains, foley catheters and central lines.

The medical tube of the present invention has color-coded sections. The terms “color-coded” and “color” as used herein mean any visual way of distinguishing different sections along the tube body. This includes differentiation based on color and/or luminescence such as phosphorescence or fluorescence. For example, sections of the tube can be of distinct colors such as red or green. Sections can also be luminescent sections, either alone instead of a color, or in addition to color-coded sections. Such luminescent sections are advantageous for detection of tube misplacement for example, in darkened rooms, or at night. In a darkened environment a luminescent warning or danger zone section will be visible. As used herein, the term “color” will also encompass luminescent “coloring”.

Each color of the color-coded section corresponds with one or more distinct warning or danger zones. If a medical professional, patient, or non-professional caregiver sees a color corresponding to a warning zone, that person is then aware that the medical tube may need to be adjusted or reinserted. Additionally, if a medical professional, patient, or caregiver sees either the color corresponding to a danger zone, or the colors for both a warning zone and a danger zone, that person is then aware that the medical tube is in need of immediate readjustment or reinsertion. The warning zone section may also comprise a series of color gradations or shades for one color, for example, starting as a light or pale shade darkening to a deeper shade of color (e.g., pale blue to sky blue to navy blue).

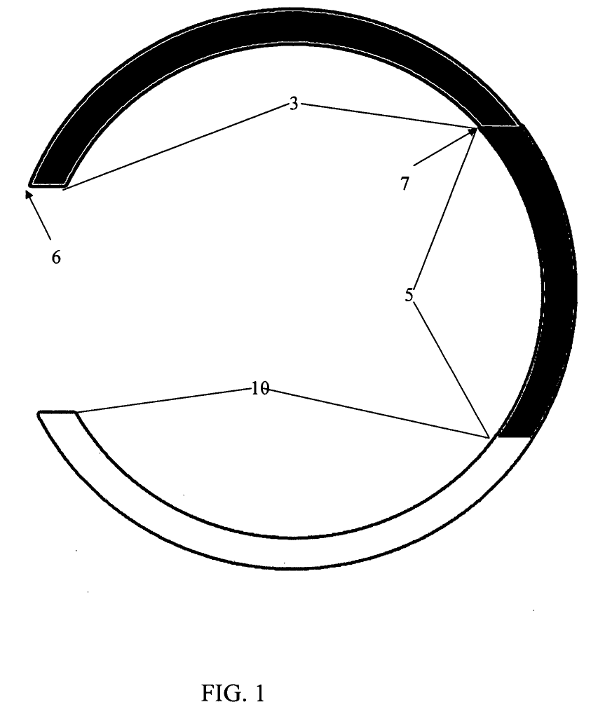

In FIG. 1, a tube body of a medical tube is depicted. This embodiment has three visually distinguishable sections: the danger zone section 3 at the distal end of the tube, the warning zone section 5, and the unlabeled proximal end of the tube body 10. The danger zone section 3 extends from the distal insertion tip 6 to a point determined by the purpose of the medical tube. For example, a feeding tube must be properly inserted into the stomach, so the danger zone section would be the length of the section of tube to be lodged in the stomach. The warning zone section 5 extends from the ending point of the danger zone section 7 to a point that is also determined by the purpose of the medical tube.

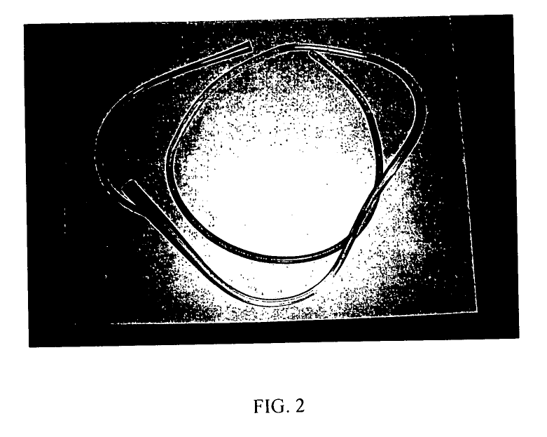

The length and/or placement of the danger and warning zone sections along the tube body are determined by the need associated with each medical tube. An example of a medical tube wherein the length of the visually distinguishable sections of the tube body is determined by the intended purpose of the tube is found in another embodiment of the present invention. FIG. 2 is a color photograph of a nasogastric tube. The nasogastric tube is a polymer tube that is inserted into the patient's nose and advanced into the stomach. In order for the nasogastric tube to function properly and to avoid discomfort and danger to the patient, the tube must be inserted beyond the esophagus and into the stomach. Because the average length of an adult human's esophagus is about 40 cm, the danger zone section 20 begins at about 40 cm 21 from the distal insertion tip 22 and extends a suitable length for easy detection 23. The warning zone section extends from the end of the danger zone section 21 to about 55 cm from the distal insertion tip 22. This length of about 15 cm is the appropriate length for a tube to be inserted within the stomach. If the nasogastric tube is inserted too far into the stomach, the tube cannot decompress the stomach, and loses clinical benefit to the patient.

The methods of producing medical tubes are well known in the art. Medical tubes are routinely made of flexible polymers. Using routine methods, such flexible polymer tubes can be manufactured to include colored/fluorescent sections. Thus, one skilled in the art would be able to safely manufacture a color-coded medical tube by, for example, impregnating the color into the tube or by adding a physiologically inert dye to the polymer during the manufacturing process.

The present invention can also be used in a method to aid in insertion of the medical tube into a patient. Referring now back to FIG. 2, the tube body comprises three visually distinct sections: the danger zone section 20 at the distal end of the tube, the warning zone section 25, and the unlabeled proximal end of the tube body 30. The danger zone section 20 is color-coded red beginning at about 40 cm 21 from the distal insertion tip 22. The warning zone section 25 is color-coded yellow from the end of the danger zone section 21 to about 55 cm from the distal insertion tip 22. When inserting the nasogastric tube, one can use the color-coded zones to assist in properly inserting the tube. For example, a medical professional would know to stop inserting the tube after both the danger 20 and warning zone section 25 colors had been inserted into the patient. This would reduce the occurrences of improper placement of nasogastric tubes, and would thus alleviate problems associated with tubes that are not inserted far enough, or that are inserted too far.

The present invention can also be used in methods of post-insertion monitoring of tube placement to aid in recognition of a tube that becomes misplaced after proper insertion. FIG. 1 depicts the tube body of a medical tube. This embodiment of the tube body of the present invention has three visually distinguishable sections: the danger zone section 3 at the distal end of the tube, the warning zone section 5, and the unlabeled proximal end of the tube body 10. When inserted properly, neither the warning 5 nor the danger 3 zone sections are visible outside the patient. Post-insertion monitoring of the medical tube can be accomplished by observing whether the color that codes for the warning zone section 5 or for the danger zone section 3 is visible. If the warning zone section 5 becomes visible, a medical professional, patient or a non-medically trained caregiver can recognize that the medical tube may be in need of adjustment or reinsertion. Thus, early warning of a potentially serious medical problem is given and the problem avoided. If the danger zone section 3 becomes visible, the medical professional, patient or caregiver can recognize that the medical tube is in immediate need of adjustment or reinsertion.

A nasogastric (NG) tube is an example of a medical tube for which color-coding would facilitate post-insertion monitoring. Nasogastric tubes are widely used in medicine and surgery for treatments that require access to the stomach. If a nasogastric tube moves substantially post-insertion, it could dislodge from the stomach and become improperly positioned in the esophagus. If a patient is fed while the nasogastric tube is improperly positioned, he/she can easily vomit, or aspirate liquid which can lead to pneumonia and even death. Although these problems are not widely publicized, every physician knows that such cases can occur.

The use of color-coded nasogastric tubes would alleviate these problems. Post-insertion monitoring is facilitated at the hospital or, if the patient is sent home with one of these tubes in place, at home. If the warning or danger zone colors are visible, even the casual observer is capable of determining that the medical tube is misplaced. Once it is determined that the medical tube is misplaced or may be misplaced, a medical professional can be called to evaluate the situation and remedy any problems.

As a result of the invention described herein, insertion of medical tubes into a patient will be a safer procedure even when performed by a medical professional, and post-insertion monitoring of tube placement will be greatly facilitated, even for a non-professional.

While this invention has been particularly shown and described with references to preferred embodiments thereof, it will be understood by those skilled in the art that various changes in form and details may be made therein without departing from the scope of the invention encompassed by the appended claims.

What is claimed is:

1. A medical tube having a tube body suitable for insertion into a patient, the tube body having at least one visibly distinguishable color-coded section.

2. The medical tube of claim 1 wherein the medical tube comprises a tube selected from the group consisting of: a nasogastric tube; a feeding tube; a chest tube; a drain; a foley catheter; and a central line.

3. The medical tube of claim 1 wherein the medical tube comprises two or more color-coded sections with one section being a visually distinguishable danger zone section and one section being a visually distinguishable warning zone section, wherein the visually distinguishable danger zone section and visually distinguishable warning zone section are different colors.

4. The medical tube of claim 1 wherein the medical tube is a nasogastric tube having a proximal end and a distal end, wherein the tube comprises at least one visually distinguishable danger zone section, wherein the danger zone section is color-coded along the tube body from the distal end to a length of about 40 cm from the distal end to the proximal end of the tube.

5. The medical tube of claim 1 wherein the medical tube is a nasogastric tube having a proximal end and a distal end, wherein the tube comprises at least one visually distinguishable danger zone section, wherein the danger zone section is color-coded along the tube body from the distal end to a length of about 40 cm from the distal end to the proximal end of the tube, and at least one visually distinguishable warning zone section, wherein the warning zone section is a different color from the danger zone section along the tube body from after the visually distinguishable danger zone section to about 55 cm from the distal end of the tube.

6. A method of inserting a medical tube into a patient, the method comprising the steps of:
a) providing a color-coded medical tube and inserting the tube into the patient, wherein the tube comprises at least one color-coded section; and
b) halting the insertion of the medical tube into the patient once the color-coded section of the medical tube has been completely inserted into the patient.

7. The method of claim 6 wherein the medical tube is a tube selected from the group consisting of: a feeding tube, a chest tube, a drain, a foley catheter and a central line.

8. A method of inserting a medical tube into a patient, the method comprising the steps of:
a) providing a color-coded medical tube and inserting the tube into the patient wherein the tube comprises at least one visually distinguishable danger zone section that is color-coded and at least one visually distinguishable warning zone section that is color-coded; and
b) halting the insertion of the medical tube into the patient once the danger zone section and the warning zone section of the medical tube have been completely inserted into the patient.

9. A method of post-insertion monitoring of the placement of a medical tube in a patient, wherein the medical tube comprises a tube body wherein the tube body has at least one visually distinguishable color-coded danger zone section, the method comprising observing the medical tube to detect the visually distinguishable danger zone section of the tube, wherein the observance of the danger zone section of the tube is an indication that the medical tube is misplaced.

10. The method of claim 9 wherein the medical tube is a tube selected from the group consisting of: a feeding tube, a chest tube, a drain, a foley catheter and a central line.

11. A method of post-insertion monitoring of the placement of a medical tube in a patient, wherein the medical tube comprises a tube body, wherein the tube body has at least one visually distinguishable color-coded danger zone section and at least one visually distinguishable color-coded warning zone section, comprising the step of observing the visually distinguishable warning zone section of the tube, wherein the observance of the warning zone section is an indication that the medical tube may be misplaced.
